SEQUENCE LISTING (4 sequences)

SEQ ID NO: 1 - protein, 136 aa, synthetic construct
  source 1..136: note = the heavy chain variable region of the MP-4 antibody
MEWIWIFLFILSGTAGVHSQVQLQQSGAELARPGASVKLSCKASGYTFTDYYINWVKQRTGQGLEWIGEIYPGSGNTYYNEKFKGKATLTADKSSSTAYMQLSSLTSEDSAVYFCAREDYGTPDYWGQGTTLTVSS

SEQ ID NO: 2 - protein, 128 aa, synthetic construct
  source 1..128: note = the light chain variable region of the MP-4 antibody
MDFQVQIFSFLLISASVIISRGQIVLTQSPAIMSASPGEKVTMTCSASSSVSYMHWYQQKSGTSPKRWIYDTSKLASGVPARFSGSGSGTSYSLTISSMEAEDAATYYCQQWSSNPRTFGGGTKLEIK

SEQ ID NO: 3 - protein, 139 aa, synthetic construct
  source 1..139: note = the heavy chain variable region of the MP-9 antibody
MAVLGLLFCLVTFPSCVLSQVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCASYYYGSSWYAMDYWGQGTSVTVSS

SEQ ID NO: 4 - protein, 127 aa, synthetic construct
  source 1..127: note = the light chain variable region of the MP-9 antibody
MNMLTQLLGLLLLWFAGGKCDIQMTQSPASQSASLGESVTITCLASQTIGTWLAWYQQKPGKSPQLLIYAATSLADGVPSRFSGSGSGTKFSFKISSLQAEDFVSYYCQQLYSTPLTFGAGTKLELK

(19) United States
(12) Patent Application Publication
(10) Pub. No.: US 2025/0092159 A1
(43) Pub. Date: Mar. 20, 2025

(54) HYBRIDOMA CELL LINE SECRETING ANTI-MICRO/NANO PLASTIC BROAD-SPECTRUM MONOCLONAL ANTIBODY AND USE THEREOF

(71) Applicant: Wuhan Textile University, Wuhan (CN)

(72) Inventors: Zhaowei Zhang, Wuhan (CN); Wenqin Wu, Wuhan (CN)

(21) Appl. No.: 18/966,197

(22) Filed: Dec. 3, 2024

(30) Foreign Application Priority Data
Oct. 15, 2024 (CN) 202411439931.1

(51) Int. Cl. C07K 16/44; G01N 33/53

(52) U.S. Cl. CPC C07K 16/44; G01N 33/5308

(57) ABSTRACT

A hybridoma cell line secreting an anti-micro/nano plastic monoclonal antibody and use thereof are provided. The hybridoma cell line includes hybridoma cell line MP-4 deposited under the Accession number of CCTCC NO: C2024262 and hybridoma cell line MP-9 under the Accession number of CCTCC NO: C2024261, which are screened by the inventor. The passage cell lines of the hybridoma cells can stably secrete the anti-broad-spectrum micro/nano plastic protein monoclonal antibody. The secreted antibody is high in potency and good in sensitivity, and can simultaneously recognize polystyrene (PS), polyvinyl chloride (PVC), polypropylene (PP), polyethylene (PE), and polyethylene terephthalate (PET), with good broad-spectrum recognition. Therefore, the hybridoma cell line provides a core raw material for establishing broad immune detection methods of microplastics and has wide application prospects.

### SEQUENCE LISTING

The sequence listing is submitted as a XML file filed via EFS-Web, with a file name of “Sequence_Listing. XML”, a creation date of Nov. 30, 2024, and a size of 6098 bytes. The sequence Listing filed via EFS-Web is a part of the specification and is incorporated in its entirety by reference herein.

### TECHNICAL FIELD

The present disclosure belongs to the technical field of biological materials and particularly relates to a hybridoma cell line secreting an anti-micro/nano plastic monoclonal antibody and use thereof.

### BACKGROUND

Microplastics have many types, large hazards, heavy pollution, and a wide range. The microplastics that pollute the environment mainly include polystyrene (PS), polypropylene (PP), polyethylene (PE), etc. Microplastics have hazards such as sublethal toxicity and cytotoxicity, posing a serious threat to human life and health.

Most of the existing microplastic detections depend on instrument analysis methods such as microscope spectroscopy and Raman spectroscopy, which is difficult to meet the needs of rapid and accurate detection. In China, microplastics are complicated in pollution environment, significant in geographical difference and multiple in pollution links, and therefore involved in the fields of environment, food, textiles, and the like. In the process of producing synthetic fibers (such as polyester fibers and nylon), some plastic particles or fibers smaller than 5 mm may be produced. For example, friction of production equipment, incomplete polymerization of raw materials, and other situations may lead to the generation of microplastics. In addition, in the textile process, such as spinning, weaving, printing dyeing, and other links, the operation of a machine, abrasion of parts, and stress application during the processing may make microplastic fibers on the surface of the textile detach from the surface of the textile. Especially for some high-strength processing processes, poor raw materials are used, which may increase the possibility of generating microplastics. When people wear textiles, the fibers on the surface of the textile are gradually worn and fall off due to body movements, friction, and others, thereby generating microplastic fibers. For example, fiber shedding is easier to occur in some areas frequently rubbed, such as cuffs, collars, and pant legs. Meanwhile, during the washing in a washing machine, the friction between clothes, the impact of water flow, and the action of detergent can cause a large number of fibers to fall off from the clothes, forming microplastic fibers. It is estimated that in a typical household washing activity, a piece of clothing can release millions of fibers. Furthermore, some clothes made of synthetic fibers, such as nylon and dacron, more easily produce microplastic fibers during the washing due to their fiber structures and features. Whereas when wasted textiles are buried, the textiles will be gradually degraded and broken in a natural environment over time to generate microplastics. These microplastics may enter environments such as soil and water with rainwash or other approaches, causing pollution. Although incineration can reduce the amount of solid waste from textiles, some smoke and ashes containing microplastics may be generated during the incineration. If these smoke and ashes are not treated well, they can also be released into the environment, causing microplastic pollution.

At present, the main detection methods of microplastics include a visual inspection method, a microscopy method, an electron microscopy method, a Raman spectroscopy method, an infrared spectroscopy method, a thermal analysis method, a pyrolysis gas chromatography-mass spectrometry method, a laser infrared imaging method, and an optical thermal infrared technology. However, some of these detection technologies can only make qualitative judgments, some detection technologies have low detection sensitivity, and some detection technologies rely on large-scale laboratory instruments for detection, thus the real-time monitoring for pollution distribution and environmental effects of the microplastics in the environment is difficult. Therefore, there is an urgent need to study high-sensitivity and rapid detection methods of microplastics in complex matrices. Since microplastics have many types and different properties, there is a certain challenge in developing broad-spectrum antibodies capable of recognizing multiple different types of microplastics. Therefore, it is urgent to provide a hybridoma cell line secreting an anti-micro/nano plastic monoclonal antibody and use thereof.

### SUMMARY

To solve the defects existing in the prior art, the present disclosure provides a hybridoma cell line secreting an anti-micro/nano plastic monoclonal antibody and its use thereof.

To solve the above technical problem, the present disclosure provides the following

### Technical Solution

The first objective of the present disclosure is to provide a hybridoma cell line secreting an anti-micro/nano plastic monoclonal antibody, comprising hybridoma cell line MP-4 deposited with China Center for Type Culture Collection under the Accession number of CCTCC NO: C2024262.

The second objective of the present disclosure is to provide a hybridoma cell line secreting an anti-micro/nano plastic monoclonal antibody, comprising hybridoma cell line MP-9 deposited with China Center for Type Culture Collection under the Accession number of CCTCC NO: C2024261.

The third objective of the present disclosure is to provide a monoclonal antibody, the monoclonal antibody being secreted by the hybridoma cell line MP-4 or the hybridoma cell line MP-9, and the monoclonal antibody broadly recognizing micro/nano plastics.

Preferably, the micro/nano plastic comprises at least one of PS, polyvinyl chloride (PVC), polyethylene terephthalate (PET), PE, and PP.

The fourth objective of the present disclosure is to provide a kit, the kit comprising the monoclonal antibody secreted by the hybridoma cell line MP-4 or the hybridoma cell line MP-9.

The fifth objective of the present disclosure is to provide the use of the monoclonal antibody in preparing a reagent for detecting micro/nano plastics.

Compared with the prior art, the present disclosure has the following beneficial effects:

The passage cell lines of the hybridoma cells provided by the present disclosure can stably secrete a monoclonal antibody against a broad-spectrum micro/nano plastic protein, and the secreted antibody is high in potency and good in specificity and can be applied to kit detection, thereby significantly improving the sensitivity of kit detection.

The hybridoma cell line provided by the present disclosure can accurately detect small amounts of microplastics in the environment. Whether in water, soil, or atmosphere, even though the concentration of microplastics is low, the broad-spectrum antibody can effectively recognize and bind to microplastics, thereby providing researchers with more accurate detection results.

The present disclosure can more accurately detect microplastics in the environment. No matter whether in the water body, soil, or atmosphere, even though the concentration of microplastics is low, the broad-spectrum antibody can effectively recognize and bind microplastics, thereby providing more accurate detection results for researchers.

The present disclosure can detect multiple different types of microplastics. Since microplastics have many types and different shapes, sizes, and chemical compositions, the broad-spectrum antibody can recognize microplastics made of different materials, such as polyethylene and polypropylene, thereby significantly enlarging the coverage range of detection.

### Collection of Cells:

The hybridoma cell line secreting the anti-micro/nano plastic monoclonal antibody provided by the present disclosure comprises hybridoma cell line MP-4 and hybridoma cell line MP-9. The hybridoma cell line MP-4 and hybridoma cell line MP-9 are screened by the inventor of the present disclosure. The hybridoma cell line MP-4 and the hybridoma cell line MP-9 are deposited with China Center for Type Culture Collection under the Accession numbers of CCTCC NO: C2024262 and CCTCC NO: C2024261 respectively on Aug. 14, 2024, and the address of the collection unit is 299 Bayi Road, Wuchang District, Wuhan City, Hubei Province, on campus of Wuhan University.

### BRIEF DESCRIPTION OF THE DRAWINGS

FIG. 1 is a schematic diagram showing sodium dodecyl sulfate-polyacrylamide gel electrophoresis (SDS-PAGE) of PS-bovine serum albumin (BSA) and PS-keyhole limpet hemocyanin (KLH) in example 1 of the present disclosure;

FIG. 2 shows an acquisition process of antibody variable regions in example 5 of the present disclosure; and

FIG. 3 shows detection results in example 6 of the present disclosure.

### DETAILED DESCRIPTION OF THE EMBODIMENTS

Next, preferred embodiments of the present disclosure will be illustrated in combination with accompanying drawings, it should be understood that preferred embodiments described here are only used for illustrating and explaining the present disclosure, but not limiting the present disclosure.

### Example 1: Coupling of Microplastic (PS-CHOOH) and Coupling Protein (BSA/KLH)

1. Microplastics, polylink coupling buffer, polylink ethyl-[3-(dimethylamino) propyl]-carbodiimide hydrochloride (EDAC), and polylink wash/storage buffer were cooled to room temperature.

2. 12.5 mg of microplastics were added into a 1.5 mL centrifuge tube and then centrifuged for 5-10 min at 500-1000 g, and a supernatant was discarded; 0.4 mL of polylink coupling buffer was added for resuspension followed by centrifugation again, and then the supernatant was discarded; 0.17 mL of polylink coupling buffer was added again for resuspension.

3. 10 mg of polylink EDAC was dissolved into 50 μL of polylink coupling buffer to prepare a 200 mg/mL EDAC solution. Note: the solution was prepared when using.

4. 20 μL of EDAC solution was added into 0.17 mL of polylink coupling buffer suspension to be fixed on a rotator to be evenly mixed for 15 min at room temperature for activation.

5. 200-500 μg of coupling protein (BSA/KLH) was added and fixed on the rotator to be evenly mixed for 30-60 min at room temperature (the protein was dissolved with the polylink coupling buffer until 1-5 mg/mL).

6. The coupling protein (BSA/KLH) was centrifuged for 10 min at 500-1000 g, the supernatant was sucked away, and then the amount of the coupling protein (BSA/KLH) was measured.

7. 0.4 mL of polylink wash/storage buffer was added for resuspension.

8. Steps 6 and 7 were repeated, and the precipitate at the bottom of the tube was PS-BSA or PS-KLH and finally stored at 4° C.

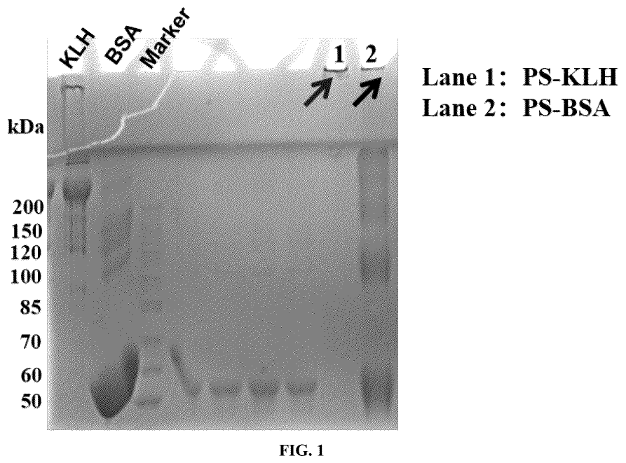

9. SDS-PAGE analysis (4% spacer gel and 7.5% separating gel) was performed. The detection results of reduced SDS-PAGE are shown in FIG. 1, proving that PS-KLH is successfully coupled with PS-BSA.

### Example 2: Preparation of Monoclonal Antibody

6 to 8-week-old female Balb/c mice were selected and immunologically injected with a prepared PS-BSA artificial antigen. Adult Balb/c mice received primary immunization through subcutaneous (in an area between shoulders) administration and were subjected to booster immunization every 2 weeks. Blood samples were collected at week 0 (before immunization), week 8 (2 weeks after the fourth booster jab), week 10 (before the third booster jab), week 12, and week 14, then serum isolation was performed to obtain B lymphocytes, and RNA was extracted and transcripted into a cDNA library. Amplification was performed again through cDNA to obtain a specific gene fragment, and the specific gene fragment was recombined onto a bacteriophage to achieve the construction of a nano antibody gene library. Subsequently, specific antibody screening was performed to obtain an antibody meeting requirements, and the PS-BSA artificial antigen was fixed on a carrier and then interacted with the bacteriophage library. Then, the surface of the carrier was washed to remove unbound or non-specific bound bacteriophages, and then their binding was destroyed with a strong acid, thereby obtaining a positive bacteriophage solution. After 2-3 rounds were repeated, the monoclonal antibody meeting the requirements was obtained.

Through the injection of the PS-BSA artificial antigen, an immunization scheme for mice is shown in Table 1, the specific response of the PS-BSA artificial antigen on the immune system of Balb/c mice was observed, and an activation mechanism of an antibody-specific immune response was explored.

TABLE 1
Immunization scheme for mice

| | | | | | Interval |
|---|---|---|---|---|---|
| | | | | | time |
| | | | | | distanced |
| | | | | | from |
| | | | | | the last |
| | | | | Dose | immuni- |
| Method | Antigen | Route | Adjuvant | (μg) | zation |
| The first | PS-BSA | Subcutaneous | Complete | 100 μg/ | 0 |
| immunization | | | | mouse | |
| The second | PS-BSA | Subcutaneous | Incomplete | 50 μg/ | 2 weeks |
| immunization | | | | mouse | |
| The third | PS-BSA | Subcutaneous | Incomplete | 50 μg/ | 2 weeks |
| immunization | | | | mouse | |
| The fourth | PS-BSA | Subcutaneous | Incomplete | 50 μg/ | 2 weeks |
| immunization | | | | mouse | |
| The fifth | PS-BSA | Subcutaneous | Incomplete | 50 μg/ | 2 weeks |
| immunization | | | | mouse | |

ELLSA tail blood potency testing

| | | | | |
|---|---|---|---|---|
| Shock | Intraperitoneal, | — | 50 | 3 days |
| immunization | tail vein, or | | | before the |
| | intrasplenic | | | fusion |

### Example 3: Subtype Analysis and Potency Testing of Monoclonal Antibody MP-4

Subtype analysis of monoclonal antibody: the subtype of the antibody MP-4 generated by 1 hybridoma cell line secreting specific monoclonal antibodies was identified. The results show that MP-4 is IgG2b.

Potency testing of a monoclonal antibody: a plate was coated with 1 μg/ml PS-KLH, each antibody was subjected to gradient dilution (1:100, 1:500, 1:2500, 1:12500, 1:32500 and 1:612500), and goat anti-mouse IgG-HRP was added to undergo 1w-fold dilution to ensure the potency of the purified MP-4 monoclonal antibody (the results are as shown in Table 2).

TABLE 2
PS-BSA mouse five-immune tail blood potency testing
Coat the plate: PS-KLH 1 μg/ml
Second antibody: goat anti-mouse IgG-HRP
1 w-fold dilution

| | Immunized | Immunized | Immunized |
|---|---|---|---|
| Dilution | mouse-1 | mouse-2 | mouse-3 |
| fold | 1 | 2 | 3 |
| 100 | (+) | (+) | (+) |
| 500 | (+) | (+) | (+) |
| 2500 | (+) | (+) | (+) |
| 12500 | 2.137 | 3.221 | 3.145 |
| 32500 | 0.706 | 0.891 | 0.983 |
| 612500 | 0.278 | 0.397 | 0.432 |
| PBS | 0.068 | 0.098 | 0.103 |

The plate was coated with 1 μg/ml PS-KLH, and each antibody was subjected to gradient dilution (1:100, 1:500, 1:2500, 1:12500, 1:32500, and 1:612500), and goat anti-rabbit IgG-HRP was added to undergo 1w-fold dilution to ensure the potency of the purified MP-4 monoclonal antibody (the results are as shown in Table 3).

TABLE 3
PS Rabbit five-immune serum potency testing
Coat the plate: PS-KLH 1 μg/ml
Second antibody: goat anti-rabbit IgG-HRP 1 w-fold dilution

| | Immunized | Immunized | Unimmunized | Unimmunized |
|---|---|---|---|---|
| Dilution | rabbit-1 | rabbit-2 | rabbit-1 | rabbit-2 |
| fold | 1 | 2 | 3 | 4 |
| 100 | 1.894 | 1.639 | 0.045 | 0.048 |
| 500 | 1.769 | 1.589 | 0.108 | 0.038 |
| 2500 | 1.543 | 1.498 | 0.037 | 0.073 |
| 12500 | 1.482 | 1.390 | 0.116 | 0.184 |
| 32500 | 1.239 | 1.265 | 0.149 | 0.162 |
| 612500 | 0.561 | 0.491 | 0.103 | 0.029 |
| PBS | 0.098 | 0.109 | 0.086 | 0.109 |

### Example 4: Subtype Analysis and Potency Testing of Monoclonal Antibody MP-9

Subtype analysis of monoclonal antibody: the subtype of the antibody MP-9 generated by 1 hybridoma cell line secreting specific monoclonal antibodies was identified. The results show that MP-9 is IgG2b.

Potency testing of a monoclonal antibody: a plate was coated with 1 μg/ml PS-KLH, each antibody was subjected to gradient dilution (1:100, 1:500, 1:2500, 1:12500, 1:32500 and 1:612500), and goat anti-mouse IgG-HRP was added to undergo 1w-fold dilution to ensure the potency of the purified MP-9 monoclonal antibody (the results are as shown in Table 4).

TABLE 4
PS-BSA mouse five-immune tail blood potency testing
Coat the plate: PS-KLH 1 μg/ml
Second antibody: goat anti-mouse IgG-HRP 1 w-fold
dilution

| | Immunized | Immunized | Immunized |
|---|---|---|---|
| Dilution | mouse-1 | mouse-2 | mouse-3 |
| fold | 1 | 2 | 3 |
| 100 | (+) | (+) | (+) |
| 500 | (+) | (+) | (+) |
| 2500 | (+) | (+) | (+) |
| 12500 | 2.390 | 2.986 | 3.281 |
| 32500 | 0.715 | 0.923 | 0.860 |
| 612500 | 0.279 | 0.346 | 0.321 |
| PBS | 0.101 | 0.139 | 0.094 |

The plate was coated with 1 μg/ml PS-KLH, and each antibody was subjected to gradient dilution (1:100, 1:500, 1:2500, 1:12500, 1:32500, and 1:612500), and goat anti-rabbit IgG-HRP was added to undergo 1w-fold dilution to ensure the potency of the purified MP-9 monoclonal antibody (the results are as shown in Table 5).

TABLE 5
PS-BSA rabbit five-immune serum potency testing
Coat the plate: PS-KLH 1 μg/ml
Second antibody: goat anti-rabbit IgG-HRP 1w-fold dilution

| | Immunized | Immunized | Unimmunized | Unimmunized |
|---|---|---|---|---|
| Dilution | rabbit-1 | rabbit-2 | rabbit-1 | rabbit-2 |
| fold | 1 | 2 | 3 | 4 |
| 100 | 1.853 | 1.678 | 0.098 | 0.056 |
| 500 | 1.730 | 1.589 | 0.101 | 0.034 |
| 2500 | 1.462 | 1.432 | 0.079 | 0.094 |
| 12500 | 1.379 | 1.390 | 0.986 | 0.018 |
| 32500 | 1.207 | 1.289 | 0.303 | 0.103 |
| 612500 | 0.548 | 0.481 | 0.104 | 0.089 |
| PBS | 0.169 | 0.029 | 0.076 | 0.103 |

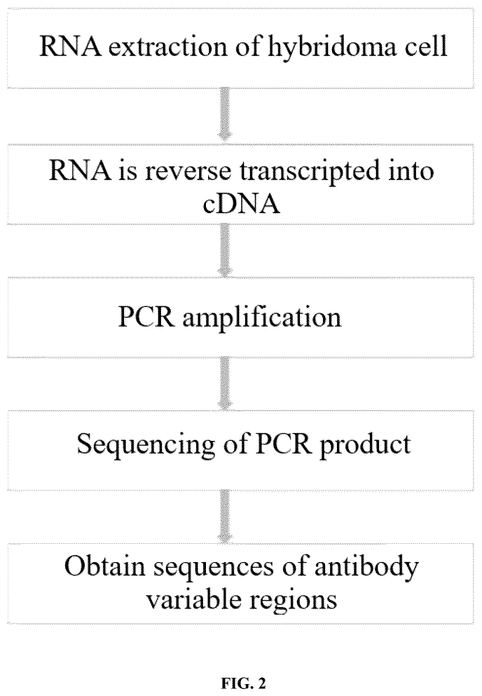

### Example 5: Sequencing of Monoclonal Antibodies MP-4 and MP-9 (the Sequences of the Variable Regions of the Antibodies were Obtained According to the Procedure in FIG. 2)

(1) The monoclonal antibody secreted by the hybridoma cell line MP-4 comprises a heavy chain variable region and a light chain variable region both of which are composed of determining cluster complementarity regions and framework regions; the determining cluster complementarity regions of the heavy chain variable region and the light chain variable region are both composed of CDR1, CDR2, and CDR3;

The amino acid sequence of CDR1 of the heavy chain variable region is as shown in position 50-54 of SEQ ID NO.1;

The amino acid sequence of CDR2 of the heavy chain variable region is as shown in position 69-85 of SEQ ID NO.1;

The amino acid sequence of CDR3 of the heavy chain variable region is as shown in position 118-125 of SEQ ID NO.1;

The amino acid sequence of CDR1 of the light chain variable region is as shown in position 46-55 of SEQ ID NO.2;

The amino acid sequence of CDR2 of the light chain variable region is as shown in position 71-77 of SEQ ID NO.2;

The amino acid sequence of CDR3 of the light chain variable region is as shown in position 110-118 of SEQ ID NO.2.

The amino acid sequence of the heavy chain variable region of the MP-4 antibody is as shown in SEQ ID NO.1:

MEWIWIFLFILSGTAGVHSQVQLQQSGAELARPGASVKLSCKASGYTFTDYYI NWVKQRTGQGLEWIGEIYPGSGNTYYNEKFKGKATLTADKSSSTAYMQLSSLTSEDS AVYFCAREDYGTPDYWGQGTTLTVSS.

The amino acid sequence of the light chain variable region of the MP-4 antibody is as shown in SEQ ID NO.2:

MDFQVQIFSFLLISASVIISRGQIVLTQSPAIMSASPGEKVTMTCSASSSVSYMH WYQQKSGTSPKRWIYDTSKLASGVPARFSGSGSGTSYSLTISSMEAEDAATYYCQQW SSNPRTFGGGTKLEIK.

(2) The monoclonal antibody secreted by the hybridoma cell line MP-9 comprises a heavy chain variable region and a light chain variable region both of which are composed of determining cluster complementarity regions and framework regions; the determining cluster complementarity regions of the heavy chain variable region and the light chain variable region are both composed of CDR1, CDR2, and CDR3;

The amino acid sequence of CDR1 of the heavy chain variable region is as shown in position 50-54 of SEQ ID NO.3;

The amino acid sequence of CDR2 of the heavy chain variable region is as shown in position 69-84 of SEQ ID NO.3;

The amino acid sequence of CDR3 of the heavy chain variable region is as shown in position 117-128 of SEQ ID NO.3;

The amino acid sequence of CDR1 of the light chain variable region is as shown in position 44-54 of SEQ ID NO.4;

The amino acid sequence of CDR2 of the light chain variable region is as shown in position 70-76 of SEQ ID NO.4;

The amino acid sequence of CDR3 of the light chain variable region is as shown in position 109-117 of SEQ ID NO.4;

The amino acid sequence of the heavy chain variable region of the MP-9 antibody is as shown in SEQ ID NO.3:

MAVLGLLFCLVTFPSCVLSQVQLKQSGPGLVQPSQSLSITCTVSGFSLTSYGVH WVRQSPGKGLEWLGVIWSGGSTDYNAAFISRLSISKDNSKSQVFFKMNSLQANDTAI YYCASYYYGSSWYAMDYWGQGTSVTVSS.

The amino acid sequence of the light chain variable region of the MP-9 antibody is as shown in SEQ ID NO.4:

MNMLTQLLGLLLLWFAGGKCDIQMTQSPASQSASLGESVTITCLASQTIGTW LAWYQQKPGKSPQLLIYAATSLADGVPSRFSGSGSGTKFSFKISSLQAEDFVSYYCQQ LYSTPLTFGAGTKLELK.

### Example 6: Use of Monoclonal Antibodies MP-4 and MP-9 for NC Membrane Detection

1. 30 μL of PS-OVA, PVC-OVA, PP-OVA, PE-OVA, and PET-OVA were streaked on an NC membrane using a filming machine in a spraying amount of 0.08 μL/cm, and 30 μL of 1 mg/mL p16 protein was taken as control to be streaked on another NC membrane. Where the p16 protein is a cyclin-dependent kinase inhibitor. The NC membrane was placed at a humidity of less than 15% and dried at 50° C. overnight.

2. The monoclonal antibodies MP-4 and MP-9 were diluted into 1 mg/mL using 20% NBS-PBS respectively, and 2 mL of antibody solution was added into a 5 mL centrifuge tube; control monoclonal antibody p16-25 was diluted into 1 mg/mL using 20% NBS-PBS, and 2 mL of antibody solution was added into a 5 mL centrifuge tube.

3. The NC membranes coated with PS-OVA, PVC-OVA, PP-OVA, PE-OVA, and PET-OVA were cut into small sections with the same width, and then respectively put in the MP-4 antibody solution and the MP-9 antibody solution; the NC membrane coated with p16 was cut into small sections with the same width, then the small sections were put in the p16-25 antibody solution, and subsequently 1 section was cut again to be put in a PS-1 antibody solution as negative control.

4. The NC membranes were incubated for 1 h at 37° C. and rinsed once with PBST.

5. Goat anti-mouse 1gG-HRP was subjected to 1 w dilution with 20% NBS-PBS, 10 mL of goat anti-mouse 1gG-HRP was taken and put in a centrifuge tube, and then the NC membranes were also put in the centrifuge tube and incubated for 30 min at 37° C.

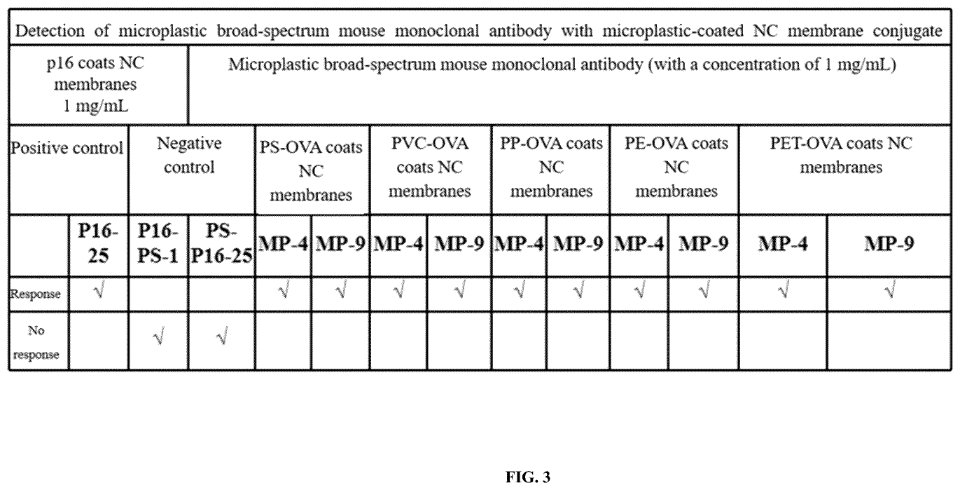

6. The NC membranes were washed 4-5 times, water was sucked away with paper, the NC membranes were completely immersed into precipitating tetramethylbenzidine (TMB) for 30 s and then taken out, water was sucked away with paper, the NC membranes were placed at a room temperature for development, and then the results were photographed on day 2 (FIG. 3).

### Example 7: Use of Monoclonal Antibody

Water and soil samples were collected from oceans, rivers, and lakes. Textile wastewater samples were collected from textile enterprises. Food samples were collected from fields or dining tables. The collected samples were subjected to pretreatment and extraction, followed by quantitative detection using a reagent kit.

The pretreatment was conducted according to the following steps:

(1) Each liquid sample such as water samples was centrifuged for 3 min at 17000×g, subsequently, a supernatant was digested and extracted, and the digested extracting solution was diluted using a sample diluent for test strip immunochromatography quantitative detection.

(2) Non-liquid samples were subjected to immunochromatographic quantitative detection after digestion treatment, microplastic extraction, and diluent dilution.

A soil sample (label testing) was taken as an example

Deionized water was added into the soil sample (1:1 w/v) and then subjected to vortex for 10 s. A slurry mixture was centrifuged for 3 min at 17000×g, and the supernatant was used as a specific matrix extract for label recovery analysis. Sample detection was performed according to the following steps:

1. 50 μL of sample pretreatment extracting solution was added into an aseptic glass tube (parallel repeated samples were set during the sample testing), and meanwhile, different volumes of microplastic working solutions were added in the same batch of tubes, the final concentrations of the added standard working solutions were 0, 1.57 μg, 3.125 μg, 6.25 μg, 12.5 μg, 25 μg and 50 μg respectively, and therefore standard curves for sample detection were established.

2. 950 μL of PBST washing buffer was then added to each tube.

3. The tubes were placed on a rotator to be rotated for 3 min at the speed of 20 r/min.

4. The tube was centrifuged for 3 min at the rotary speed of 5500 rpm to precipitate microplastic particles. The supernatant was carefully removed and discarded.

5. 1,000 μL of antibody diluent (32500-fold dilution) sample was added into each tube. The tube was subjected to a transient vortex or moved several times using a pipette to ensure the microplastics were completely mixed with the antibody solution.

6. The tube rotated for 1 h at the speed of 20 r/min at room temperature.

7. Washing steps. The tube was centrifuged for 3 min at the rotary speed of 5500 rpm to precipitate microplastic particles. The supernatant was carefully removed and discarded. 1,000 μL of washing buffer was added into each tube and the tube was subjected to transient vortex. The tube was placed on a vortex instrument to be rotated for 3 min at the rotary speed of 20 rpm.

8. Step 7 was repeated 3 times.

9. After the last washing step was completed, the tube was centrifuged for 3 min at 5500 rpm to precipitate microplastic particles. The supernatant was carefully removed and discarded.

10. 1,000 μL of goat anti-rabbit antibody labeled with horseradish peroxidase, which had been diluted 2,000 times with a PBST solution, was added into each tube. The tube was subjected to vortex for 1 min to ensure that microplastics were completely mixed with a second antibody solution.

11. The tube was placed on the vortex instrument to be rotated for 1 h at 20 rpm at room temperature.

12. Microplastics were washed 4 times using a washing buffer (steps 8-10 were repeated).

13. 100 μL of TMB substrate was added and moved several times up and down using a pipette, and then the suspension was instantly transferred to the wells of a microplate.

14. The microplate was placed at room temperature to undergo a slow shaking culture for 30 min.

15. 100 μL of 1M H₂SO₄ was added into each well for terminating the reaction.

16. The optical density (OD) value was measured at 450 nm using a microplate reader; a standard curve was plotted using Origin based on the concentration of each standard substance as a horizontal coordinate and B/BO as a longitudinal coordinate.

17. The OD value of the detected sample was substituted into the standard curve to be calculated to obtain the amount of microplastics in the sample.

Finally, it should be noted that the above descriptions are only preferred embodiments of the present disclosure, but are not used for limiting the present disclosure. Although the present disclosure has been illustrated in detail by referring to the foregoing embodiments, those skilled in the art still make modifications to the technical solutions described in the foregoing embodiments, or equivalent replacements on a part of technical features. Any modifications, equivalent replacements, improvements, and the like made within the spirit and principle of the present disclosure should be included within the scope of protection of the present disclosure.

What is claimed is:

1. A hybridoma cell line secreting an anti-micro/nano plastic broad-spectrum monoclonal antibody, comprising hybridoma cell line MP-4 deposited with China Center for Type Culture Collection under the Accession number of CCTCC NO: C2024262.

2. A hybridoma cell line secreting an anti-micro/nano plastic broad-spectrum monoclonal antibody, comprising hybridoma cell line MP-9 deposited with China Center for Type Culture Collection under the Accession number of CCTCC NO: C2024261.

3. A monoclonal antibody, the monoclonal antibody is secreted by the hybridoma cell line MP-9 according to claim 2, and the monoclonal antibody broadly recognizes micro/nano plastics.

4. The monoclonal antibody according to claim 3, wherein the micro/nano plastic comprises at least one of polystyrene, polyvinyl chloride, polyethylene terephthalate (PET), polyethylene (PE), and polypropylene (PP).

5. A kit, comprising the monoclonal antibody secreted by the hybridoma cell line MP-4 or the hybridoma cell line MP-9 according to claim 3.

6. Use of the monoclonal antibody according to claim 3 in preparing a reagent for detecting microplastics.
